(19) United States
(12) Patent Application Publication
(10) Pub. No.: US 2011/0092998 A1
(43) Pub. Date: Apr. 21, 2011

(54) BALLOON HYDRAULIC AND GASEOUS EXPANSION SYSTEM

(75) Inventors: Eran Hirszowicz, Ramat Gan (IL); Jeffrey S. Brooks, Ra'anana (IL); David Frankfurter, Ra'anana (IL)

(73) Assignee: Spatz FGIA, Inc., Jericho, NY (US)

(21) Appl. No.: 12/903,448

(22) Filed: Oct. 13, 2010

Related U.S. Application Data
(60) Provisional application No. 61/250,888, filed on Oct. 13, 2009.
(60) Provisional application No. 61/329,643, filed on Apr. 30, 2010.

(51) Int. Cl. A61F 2/04

(52) U.S. Cl. 606/192

(57) ABSTRACT

Apparatus is provided, including an intrabody balloon configured to be in a folded state during insertion into a subject's body, and to become inflated inside the subject's body. The apparatus also includes one or more bands disposed annularly around the balloon at respective longitudinal positions along the balloon. The bands are configured, while the balloon is in the folded state thereof, to secure the balloon in the folded state, and due to the balloon becoming inflated, to slide and become coupled to a portion of the apparatus. Other applications are also described.

### CROSS-REFERENCES TO RELATED APPLICATIONS

The present application:

(a) claims the priority from U.S. Provisional Application 61/250,888, entitled “Balloon hydraulic and gaseous expansion system,” to Hirszowicz, filed Oct. 13, 2009;

(b) claims the priority from U.S. Provisional Application 61/329,643, entitled, “Balloon hydraulic and gaseous expansion system,” to Hirszowicz et al., filed Apr. 30, 2010;

(c) is related to U.S. patent application Ser. No. 11/132,855, entitled “Floating Gastro-intestinal anchor,” to Brooks, filed May 18, 2005, which claims the benefit of U.S. Provisional Application 60/639,843, entitled “Intra-gastric anchoring device for weight loss balloon,” filed on Dec. 27, 2004; and

(d) is related to a PCT patent application entitled, “Balloon hydraulic and gaseous expansion system,” to Hirszowicz et al., filed on even date herewith.

All of the aforementioned applications are incorporated herein by reference.

### FIELD OF THE INVENTION

The present invention generally relates to apparatus and methods for inserting medical apparatus. Specifically, the present invention relates to apparatus and methods for insertion of a device in the gastrointestinal tract.

### BACKGROUND OF THE INVENTION

Intragastric balloons can be used to treat bariatric patients with Class 2 or Class 3 obesity (BMI>30 kg/m2) and are designed to provide short-term therapy for moderately obese individuals who need to lose weight in preparation for surgery, or as part of a dietary and behavioral modification program. Generally, placement and removal of the intragastric balloon requires the use of endoscopy. In some cases, a hospital stay of one to two days is required to fit the balloon to the specific patient.

PCT Publication WO 06/070361 to Brooks describes apparatus for use in a gastrointestinal tract of a subject. The apparatus includes a straightening rod, and a flexible tubular anchor having a distal end and an open proximal end, and sized to fit in the gastrointestinal tract. The anchor is described as comprising a material that has an elastic memory which biases the anchor toward assuming a pre-selected bent configuration. The anchor is described as being shaped so as to define a central core extending from the open proximal end toward the distal end. The anchor is configured to be straightened from the pre-selected bent configuration by insertion of the straightening rod in the central core. The apparatus further includes a device coupled to the anchor, selected from the list consisting of: a therapeutic device, and a transmitting device. Other embodiments are also described.

PCT Publication WO 07/110866 to Brooks describes apparatus for use in a stomach of a subject. The apparatus includes a balloon, adapted for placement in the stomach, and an anchor, coupled to the balloon. The anchor is adapted to prevent the balloon from passing into a duodenum of the subject. The apparatus further includes an inflation tube, coupled to the balloon to permit inflation of the balloon, and is adapted to stretch from the stomach to a mouth of the subject to facilitate inflation of the balloon. Other embodiments are also described.

PCT Application Publication WO 08/132745 to Brooks describes a method including inserting a gastric balloon coupled to an inflation tube into a stomach of a subject without using an endoscope. The method further includes non-endoscopically identifying a presence of the gastric balloon within the stomach of the subject and inflating the gastric balloon in response to the identifying. Other embodiments are also provided.

U.S. Pat. No. 6,569,173 to Blatter et al. describes compression plates and vascular anvils for anastomosis of structures including end-to-end and end-to-side anastomosis. Vascular anvils are described to cooperate in the opening of the anastomosis fenestra, engagement by a compression plate and subsequent eversion of the anastomosis fenestra contour, and also in establishing the contact engagement of the anastomosed structures. Compression plates are described as holding anastomosed structures while pemitting their dilation and keeping the anastomosis leak-proof. One of the compression plates is described as assisting in the eversion of the anastomosis fenestra contour. These compression plates and vascular anvils are described as being used with or without catheterization in minimally invasive interventions. A non-endoscopic extravascular device (hereinafter referred to as “peripheral device”) is described as being used when there is no need to use a visual aid, such as an endoscope, in a peripheral procedure. An endoscopic or peripheral device is described as carrying a compression plate device and the graft vessel, and engages the extravascular portion of the wire.

US 2002/0055757 to Torre et al. describes a space occupying device for deployment within a patient's stomach and methods of deploying and removing the device. The device is described as including an expandable member and fasteners, such as sutures, that extend at least partially through the patient's stomach wall, and that anchor the device with the patient's stomach. The device is described as being deployed and/or removed through transesophageal approaches and/or through a combination of transesophageal and transabdominal approaches.

PCT Publication WO 05/009288 and US 2005/0033331 to Burnett et al. describe methods, devices and systems to facilitate intermittent and/or partial obstruction of a pyloric valve. Devices are described as including a support portion for preventing the device from passing through the pyloric valve and a tissue engagement portion for contacting tissue adjacent the pyloric valve to obstruct the valve. Some embodiments are also described as including a positioning member extending from the tissue engagement portion for helping position the device for obstructing the valve. A retaining member is described as being optionally included on the distal end of the positioning member for further maintaining a position of the device in the stomach. Some embodiments are deliverable into the stomach through the esophagus, either by swallowing or through a delivery tube or catheter. Some embodiments are frilly reversible. Some embodiments self-expand within the stomach, while others are inflated or otherwise expanded.

PCT Publication WO 05/094257 to Birk describes a gastric balloon and method of adding and removing fluid therefrom. The gastric balloon includes a shell, a receiver, and a retractable tubing housed in the receiver and extendable from the stomach of a patient to the mouth of the patient. The shell is inflated and deflated from outside the body of the patient. The method of adding or removing fluid from the implanted gastric balloon includes steps of inserting a gastroscopic tool into the stomach of a patient and grasping an end of a retractable tubing housed in a receiver of the gastric balloon. Further steps of the method include withdrawing at least a portion of the retractable tubing from the stomach and out of a patient's mouth and adding or removing fluid from the gastric balloon via the retractable tubing withdrawn from the patient.

U.S. Pat. No. 6,183,461 to Matsuura et al. describes a drug-infusing device that is implanted into a body cavity such as a bladder. The device is implanted in an uninflated, low profile state. After insertion into the body cavity, the device is filled with a substance, such as a drug, and assumes an increased profile. After the device is filled, it is allowed to float freely within the body cavity. Alternatively, the device can be tethered to a wall of the body cavity. The device delivers the drug at a controlled rate over an extended period of time. In order to deliver the drug at a controlled rate, the device preferably has a pressure-responsive valving member. The flow resistance of the valving member is responsive to the pressure at which the drug is stored within the infusing device. The resistance of the valving member decreases as the pressure within the infusing device decreases, thereby providing a resultant controlled flow rate.

U.S. Pat. No. 5,536,274 to Neuss describes a spiral implant for organ pathways, in particular for blood vessels, which is formed from a primary spiral made of metal or a primary tube made of an elastic synthetic material whose anterior end is closed and whose cross-section at a distance of 0.5 to 2 mm from the other end of the primary spiral or primary tube is modified for a distance of 0.01 to 10 mm in order to make it possible to reposition the implant. The force required to displace the primary shape on the guide wire serving as an insertion aid amounts to 0.5N to 10N. The invention describes an implant which can be used in vascular surgery or vascular radiology in order to achieve selective blood vessel occlusion. Insertion of the implant into a blood vessel is brought about by means of an insertion catheter, with the implant being held in a rectilinear shape by a guide wire slid therein. After the implant has reached the correct position in the organ pathway it assumes a pre-formed spiral shape on the withdrawal of the insertion wire from inside it, thereby ensuring fixation of the implant in the organ pathway.

US 2007/0088380 to Hirszowicz et al. describes a balloon catheter comprising: a hollow inner shaft disposed within a hollow outer shaft; a balloon attached at its proximal end to said outer shaft and at its distal end to said inner shaft; wherein the inner shaft is constructed such that following radial expansion of the balloon to a first expanded state, said inner shaft is capable of responding to further longitudinal expansion of the balloon to a second expanded state by increasing its length from a resting value, and of responding to subsequent partial deflation back to said first expanded state by reducing its length back to said resting value.

US 2007/0083224 to lively describes a gastric bariatric balloon includes an upper balloon chamber for sealing against the fundus, a lower balloon chamber for sealing against the antrum and occupying the body of the stomach, and a supplementary chamber for filling at least a portion of the fundus. A tubular inlet is proximal the esophagus entry for receiving a hollow needle. This inlet is filled with a self-sealing material so that when punctured by the needle the material closes the needle hole upon withdrawal of the needle. Spaced apart voids in the material enable fluid interchange between the needle and each of the three chambers separately depending upon the depth of the needle within the inlet tube. Safety arms are mounted on the upper balloon chamber so as to prevent passage of the balloon through the pylorus in case of sudden deflation.

The following patents and patent applications may be of interest:

British Patent Application Publication GB 2139902 to Celestin et al.

Canadian Patent Application Publication 2483335 to Byrum et al.

Canadian Patent Application Publication CA 1233387 to Garren et al.

Canadian Patent Application Publication CA 2068715 to Kuzmak

European Patent Application Publication EP 0246999 to Eshel et al.

European Patent Application Publication EP 1342458 to Creusy et al.

German Patent Application Publication DE 3227585 to Woerner

German Patent Application Publication DE 3310234 to Husfeldt

German Patent Application Publication DE 3326061 to Woerner

German Patent Application Publication DE 3540936 to Stricker et al.

Italian Patent IT 1235492 to Frimberger et al.

PCT Publication WO 01/66166 to Birk

PCT Publication WO 02/40081 to Bales et al.

PCT Publication WO 03/055420 to Lointier et al.

PCT Publication WO 03/095015 to Alverdy

PCT Publication WO 04/014237 to Gannoe et al.

PCT Publication WO 04/089262 to Paganon

PCT Publication WO 04/105622 to Ritchie

PCT Publication WO 05/039457 to Paganon et al.

PCT Publication WO 05/107641 to Chen et al.

PCT Publication WO 86/06611 to Kullas et al.

PCT Publication WO 87/00034 to Taylor

PCT Publication WO 98/56321 to Pier et al.

U.S. Pat. No. 4,315,509 to Smit

U.S. Pat. No. 4,416,267 to Gwen et

U.S. Pat. No. 4,485,805 to Foster

U.S. Pat. No. 4,694,827 to Weiner et al.

U.S. Pat. No. 4,696,288 to Kuzmak et al.

U.S. Pat. No. 4,738,667 to Galloway

U.S. Pat. No. 4,739,758 to Lai et al.

U.S. Pat. No. 4,899,747 to Gauen et al.

U.S. Pat. No. 4,908,011 to Jacobsen et al.

U.S. Pat. No. 4,925,446 to Garay et al.

U.S. Pat. No. 5,052,998 to Zimmon

U.S. Pat. No. 5,084,061 to Gau et al.

U.S. Pat. No. 5,129,915 to Cantenys

U.S. Pat. No. 5,234,454 to Bangs

U.S. Pat. No. 5,433,216 to Sugrue et al.

U.S. Pat. No. 6,364,868 to Ikeguchi

U.S. Pat. No. 6,656,194 to Gannoe et al.

U.S. Pat. No. 6,743,198 to Tihon

U.S. Pat. No. 6,746,460 to Gannoe et al.

U.S. Pat. No. 7,004,176 to Lau

U.S. Pat. No. 7,033,384 to Gannoe, et al.

US 2003/0191492 to Gellman et al.

US 2003/158569 to Wazne

US 2003/171768 to MeGhan

US 2004/0044357 to Gannoe et al.

US 2004/0267378 to Gazi et al.

US 2004/059289 to Garza et al.

US 2004/088008 to Gannoe et al.

US 2004/093091 to Gannoe et al.

US 2005/0033345 to DeLegge

US 2005/004430 to Lee et al.

US 2005/0055039 to Burnett et al.

US 2005/0070937 to Jambor et al.

US 2005/0085923 to Levine et al.

US 2005/0192614 to Binmoeller

US 2005/0228504 to Demarais

US 2005/0267595 to Chen et al.

US 2005/0267596 to Chen et al.

US 2006/0020278 to Burnett et al.

US 2006/0190019 to Gannoe et al.

US 2006/0206064 to Kagan et al.

US 2006/0271088 to Alfrhan

The following articles may be of interest:

Borowski A et al., “Minimally invasive, nonendoscopic saphenectomy for coronary bypass surgery,” J Card Surg 16(6):484-6 (2001)

Kadakia S C et al., “Esophageal dilation with polyvinyl bougies using a marked guidewire without the aid of fluoroscopy,” Am J Gastro 88:1381-86 (1993)

Fleischer D E et al., “A marked guidewire facilitates esophageal dilation,” Am J Gastro 84:359-61 (1989)

Dumon J R et al., “A new method of esophageal dilation using Savary-Gilliard bougies,” Gastro Endosc 31:379-82 (1985)

Werth et al., “A safe and quick method for endoscopic retrieval of multiple gastric foreign bodies using a protective sheath,” Surg Gynecol Obstet 171(5):419-20 (1990)

### SUMMARY OF EMBODIMENTS

For some applications of the present invention, a gastric balloon is inserted into a subject's stomach in a folded state. During the insertion of the balloon into the subject's stomach, one or more (e.g., two or more) bands are disposed annularly around the balloon at respective longitudinal positions along the balloon. The bands secure the balloon in the folded state during insertion of the balloon into the stomach. When the balloon is inside the subject's stomach, the balloon is inflated via an inflation tube. During the inflation of the balloon, the bands slide proximally, advance toward, and/or become coupled to a proximal portion of the balloon. For some applications, the bands are configured to control inflation of the balloon, for example, such that a distal portion of the balloon expands first, and subsequently, successive proximal portions of the balloon expand until a proximal portion of the balloon expands.

(In this context, in the specification and in the claims, “proximal” means closer to the orifice through which system 18 is originally placed into the stomach of the subject, and “distal” means further from this orifice.)

Typically, the bands being disposed around the balloon during insertion of the balloon into the subject's stomach facilitate insertion of the balloon into the stomach without a sheath, a sleeve, or another covering device needing to be placed around the balloon. Further typically, the bands are disposed around the balloon such that the bands do not separate from the implanted apparatus, as a result of inflation of the balloon.

In its folded state, the distal end of the balloon is typically tapered so as to facilitate atraumatic advancement of the balloon through the esophagus of the subject.

There is therefore provided, in accordance with some applications of the present invention, apparatus, including:

an intrabody balloon configured to be in a folded state during insertion into a subject's body, and to become inflated inside the subject's body; and

one or more bands disposed annularly around the balloon at respective longitudinal positions along the balloon, the bands configured:
while the balloon is in the folded state thereof, to secure the balloon in the folded state, and
due to the balloon becoming inflated, to slide and become coupled to a portion of the apparatus.

In some applications of the present invention, the one or more bands include a plurality of bands, and a band that is disposed most distally along the balloon with respect to other bands of the plurality of bands is coupled to at least one other band of the plurality of bands.

In some applications of the present invention, the portion of the apparatus includes a portion of the balloon, and the one or more bands are configured to be coupled to the portion of the balloon.

In some applications of the present invention, the balloon includes a gastric balloon.

In some applications of the present invention, the balloon includes a 400-1000 cc balloon.

In some applications of the present invention, each band of the one or more bands has an inner diameter of between 5 and 30 mm in the folded state of the balloon.

In some applications of the present invention, each band has an inner diameter of between 10 and 20 mm in the folded state of the balloon.

In some applications of the present invention, each band is configured to expand to between 15 and 150 mm during the inflation of the balloon.

In some applications of the present invention, the one or more bands includes two or more bands.

In some applications of the present invention, the one or more bands includes 5 to 7 bands.

In some applications of the present invention, the apparatus further includes an anchor coupled to a proximal portion of the balloon and configured to anchor the balloon inside the subject's body, the bands are configured to become coupled to the anchor responsively to the inflating.

In some applications of the present invention, the bands are configured to slide proximally toward the proximal portion of the balloon.

In some applications of the present invention, the portion of the apparatus includes the proximal portion of the balloon, and the one or more bands are configured to be coupled to the proximal portion of the balloon.

In some applications of the present invention, the portion of the apparatus includes a portion of the anchor, and the one or more bands are configured to be coupled to the portion of the anchor.

In some applications of the present invention, the one or more bands include a plurality of bands disposed annularly around the balloon, at respective longitudinal positions along the balloon.

In some applications of the present invention, the plurality of bands are expandable responsively to the inflation of the balloon.

In some applications of the present invention, the plurality of bands facilitate controlled inflation of the balloon when, responsively to increasing levels of inflation of the balloon, a distal-most band of the plurality of bands is displaced and each remaining band of the plurality of bands slides proximally, in succession, to expose and facilitate unfolding of successive portions of the balloon.

In some applications of the present invention, the apparatus further includes a catheter coupled at a distal portion thereof to a proximal portion of the balloon.

In some applications of the present invention, the bands are configured to slide proximally toward the proximal portion of the balloon.

In some applications of the present invention, the portion of the apparatus includes the proximal portion of the balloon, and the one or more bands are configured to be coupled to the proximal portion of the balloon.

In some applications of the present invention, the portion of the apparatus includes a portion of the catheter that is proximal to the proximal portion of the balloon, and the one or More bands are configured to be coupled to the portion of the catheter that is proximal to the proximal portion of the balloon.

In some applications of the present invention, in the folded state, a distal end of the balloon is rounded.

In some applications of the present invention, the distal portion of the catheter is disposed within the balloon, and a proximal portion of the catheter extends away from the balloon.

In some applications of the present invention, the balloon has an opening that is sealed around a portion of the catheter.

In some applications of the present invention, the portion of the apparatus includes an inflation tube, the balloon being configured to be inflated via the inflation tube, the bands are configured to become coupled to the inflation tube responsively to the inflating.

In some applications of the present invention, the inflation tube is configured to be decoupled from the balloon and to remove the bands from a body of the subject by the inflation tube being removed from the subject's body.

In some applications of the present invention, the apparatus is configured to be positioned in an antrum of a stomach of a subject such that the apparatus to some extent interferes with natural antral contractions of the antrum.

There is additionally provided, in accordance with some applications of the present invention, a method, including:

providing apparatus that includes an intrabody balloon that is configured to be in a folded state during insertion into a subject's body, and to become inflated inside the subject's body; and

placing one or more bands annularly around the balloon at respective longitudinal positions along the balloon, such that:
while the balloon is in the folded state thereof, the bands secure the balloon in the folded state, and
due to the balloon becoming inflated, the bands slide proximally and become coupled to a portion of the apparatus.

In some applications of the present invention, placing the one or more bands annularly around the balloon includes facilitating controlled inflation of the balloon when the balloon is inflated.

In some applications of the present invention, placing the one or more bands annularly around the balloon includes facilitating successive unfolding of the balloon when the balloon is inflated.

In some applications of the present invention, the portion of the apparatus includes a portion of the balloon.

In some applications of the present invention, placing the one or more bands annularly around the balloon includes placing one or more bands that are configured to expand due to the balloon becoming inflated.

In some applications of the present invention, providing the apparatus that includes the intrabody balloon includes providing apparatus that includes a gastric balloon.

In some applications of the present invention, providing the apparatus that includes the gastric balloon includes providing apparatus that includes a 400-1000 cc balloon.

In some applications of the present invention, placing the one or more bands annularly around the balloon includes placing one or more bands which each has an inner diameter of between 5 and 30 mm in the folded state of the balloon.

In some applications of the present invention, placing the one or more bands annularly around the balloon includes placing one or more bands which each has an inner diameter of between 10 and 20 mm in the folded state of the balloon.

In some applications of the present invention, placing the one or more bands annularly around the balloon includes placing one or more bands which each is configured to expand to between 15 and 150 mm during the inflation of the balloon.

In some applications of the present invention, the method further includes coupling a proximal portion of the balloon to a distal portion of a catheter.

In some applications of the present invention, coupling the proximal portion of the balloon to the distal portion of the catheter includes coupling the proximal portion of the balloon to the distal portion of a catheter in a manner in which the distal portion of the catheter is disposed within the balloon and a proximal portion of the catheter extends away from the balloon.

In some applications of the present invention, the portion of the apparatus includes a portion of the proximal portion of the catheter.

In some applications of the present invention, the portion of the apparatus includes the proximal portion of the balloon.

In some applications of the present invention, the method further includes coupling an inflation tube to a portion of the balloon.

In some applications of the present invention, the portion of the apparatus includes a portion of the inflation tube.

In some applications of the present invention, placing one or more bands annularly around the balloon includes placing two or more bands annularly around the balloon.

In some applications of the present invention, placing two or more bands annularly around the balloon includes placing 5 to 7 bands annularly around the balloon.

In some applications of the present invention, the method further includes coupling a distal-most band of the two or more bands to another band of the one or more bands.

There is also provided, in accordance with some applications of the present invention a method, including:

inserting apparatus including an intrabody balloon into a body of a subject, while the balloon is a folded state, and while one or more bands are annularly around the balloon at respective longitudinal positions along the balloon, such that the bands secure the balloon in the folded state; and

when the balloon is disposed inside the subject's body, causing the bands to slide proximally and become coupled to a portion of the apparatus, by inflating the balloon.

In some applications of the present invention, causing the bands to slide proximally and become coupled to a portion of the apparatus includes causing the bands to become coupled to a portion of the balloon.

In some applications of the present invention, the method further includes anchoring the balloon inside the subject's body using an anchor, causing the bands to slide proximally and become coupled to a portion of the apparatus includes causing the bands to become coupled to the anchor.

In some applications of the present invention, inserting the apparatus including the intrabody balloon into the body of the subject includes inserting apparatus including a gastric balloon, via an esophagus of the subject, into a stomach of the subject, and inflating the balloon includes inflating the balloon when the balloon is inside the stomach of the subject.

In some applications of the present invention, inserting the apparatus including the gastric balloon includes inserting apparatus including a 400-1000 cc balloon.

In some applications of the present invention, inflating the balloon includes expanding the one or more bands.

In some applications of the present invention, each band of the one or more bands has a resting inner diameter of between 5 and 30 mm in the folded state of the balloon.

In some applications of the present invention, each band of the one or more bands has a resting inner diameter of between 10 and 20 mm in the folded state of the balloon.

In some applications of the present invention, expanding the one or more bands includes expanding the one or more bands to between 15 and 150 mm during the inflating.

In some applications of the present invention, the method further includes coupling a proximal portion of the balloon to a distal portion of a catheter.

In some applications of the present invention, coupling the proximal portion of the balloon to the distal portion of the catheter includes coupling the proximal portion of the balloon to the distal portion of a catheter in a manner in which the distal portion of the catheter is disposed within the balloon and a proximal portion of the catheter extends away from the balloon.

In some applications of the present invention, the portion of the apparatus includes a portion of the proximal portion of the catheter.

In some applications of the present invention, the portion of the apparatus includes the proximal portion of the balloon.

In some applications of the present invention, the one or more bands includes two or more bands.

In some applications of the present invention, the one or more bands includes 5 to 7 bands.

In some applications of the present invention, the one or more bands includes a plurality of bands, and inflating the balloon includes:

displacing a distal-most band of the plurality of bands; and sliding proximally each remaining band of the plurality of bands in succession, responsively to increasing levels of inflation of the balloon.

In some applications of the present invention, sliding proximally each remaining band of the plurality of bands in succession includes controlling inflation of the balloon.

In some applications of the present invention, sliding proximally each remaining band of the plurality of bands in succession includes unfolding successive portions of the balloon.

In some applications of the present invention, inflating the balloon includes inflating the balloon via an inflation tube, and causing the bands to slide proximally and become coupled to a portion of the apparatus includes causing the bands to become coupled to the inflation tube.

In some applications of the present invention, the method further includes, subsequent to the bands becoming coupled to the inflation tube, decoupling the inflation tube from the balloon and removing the bands from a body of the subject by removing the inflation tube from the subject's body.

The present invention will be more fully understood from the following detailed description of embodiments thereof, taken together with the drawings, in which:

### BRIEF DESCRIPTION OF THE DRAWINGS

FIG. 1 is a schematic illustration of a gastric balloon coupled to a distal end of a catheter, being inserted into a subject's stomach, two or more bands being disposed around the balloon, in accordance with some applications of the present invention;

FIG. 2 is a schematic illustration of the positioning of the balloon in the subject's stomach, in accordance with some applications of the present invention;

FIGS. 3-4 are schematic illustrations of the deformation of the catheter into an anchor for anchoring the balloon in the subject's stomach, in accordance with some applications of the present invention;

FIG. 5 is a schematic illustration of the distal end of the balloon being inflated, thereby causing the most distal band of the bands to slide off the balloon, in accordance with some applications of the present invention;

FIGS. 6-9 are schematic illustrations of respective bands being slid proximally along the balloon at respective stages of the inflation of the balloon, in accordance with some applications of the present invention; and

FIG. 10 is a schematic illustration of the balloon following inflation, in accordance with some applications of the present invention.

### DETAILED DESCRIPTION OF EMBODIMENTS

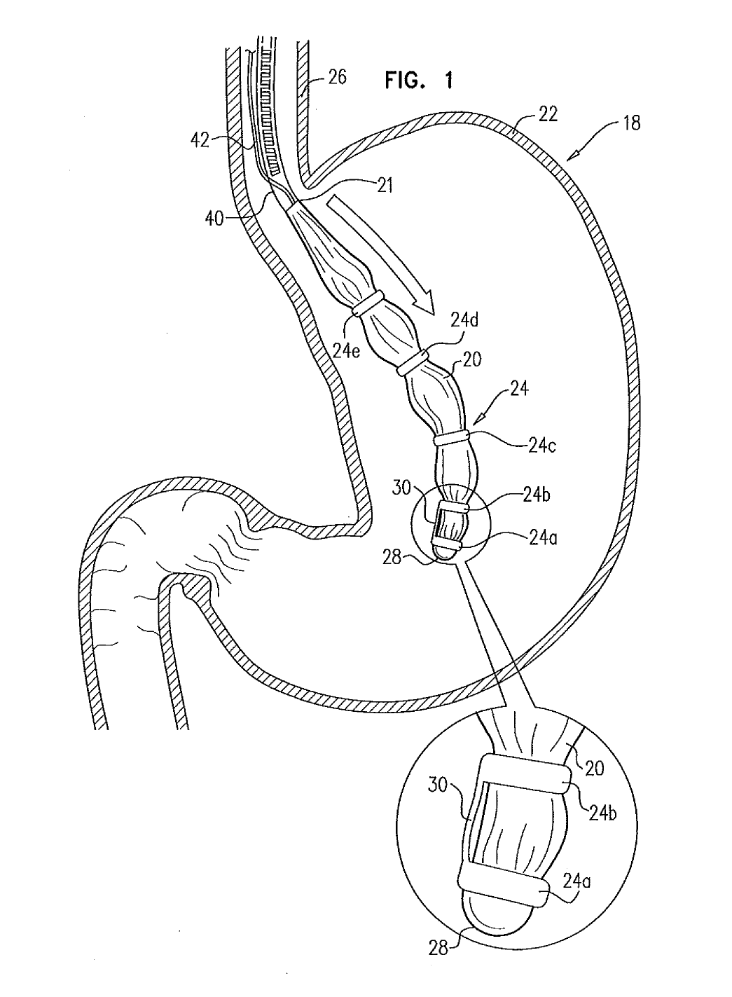

FIG. 1 is a schematic illustration of a system 18 comprising an intrabody balloon 20 (e.g., a gastric balloon, as shown) being inserted into a subject's stomach 22, one or more (e.g., two or more) bands 24 being disposed around the balloon, in accordance with some applications of the present invention. As shown, balloon 20 is typically inserted into the subject's stomach, via the subject esophagus 26, while the balloon is in a folded, deflated state. In a resting state of balloon 20 (i.e., in the absence of a force applied to balloon 20 in order to expand and inflate it), bands 24 secure balloon 20 in the folded shape, thereby facilitating passage of balloon 20 through the subject's esophagus. Bands 24 typically facilitate the passage of balloon 20 through the subject's esophagus, even in the absence of a sheath, a sleeve, or another covering device around the balloon.

Typically, four or more bands (e.g., five to seven bands) are disposed around the balloon, as is appropriate based upon the length of the balloon. Further typically, the bands are made of a stretchable material, such as silicone. In the resting state of balloon 20, bands 24 have a resting inner diameter of between 5 and 30 mm (e.g., between 10 and 20 mm), which enables bands 24 to keep balloon 20 in a folded state. Upon expanding and inflating balloon 20, bands 24 stretch and expand to an inner diameter of between 15 and 150 mm (i.e., 1.5 to 5 times the inner diameter of bands 24 in the resting state of balloon 20) in order to accommodate the expanding and inflating of balloon 20. During the expanding of balloon 20, bands 24 expand and slide with respect to an external surface of balloon 20. For some applications of the present invention, balloon 20 and/or bands 24 are coated with an anti-sticking agent, e.g., Parylene, which prevents bands 24 from sticking to balloon 20.

For some applications, the tip of a distal end 28 of the balloon is tapered or rounded, while the balloon is in the folded state thereof. Typically, the tapering of the distal tip of the balloon allows easier intubation of the proximal esophagus, and also may reduce the likelihood of the distal tip of the balloon damaging the esophageal wall, during passage of the balloon through the subject's esophagus.

For some applications, the most distal of bands 24, band 24a, is coupled to the next most distal band 24b (i.e., the band that is immediately proximal to the distal-most band) or to any other one of bands 24, for example via an optional bridge 30, as shown. The bridge prevents the most distal band from falling off the distal tip of the balloon during inflation of the balloon, when the most distal band is displaced, as described in further detail hereinbelow. It is to be noted, however, that for some applications of the present invention, none of bands 24 are coupled together (i.e., all of bands 24 are distinct from each other) by bridge 30 (or any other bridging apparatus). For such an application in which none of bands 24 are coupled together, one or more bands 24 may slide off and be decoupled from balloon 20 and system 18 and pass into stomach 22. These bands which are decoupled from system 18 then pass through the digestive system of the subject.

For applications in which balloon 20 comprises a gastric balloon, the balloon is capable of being inflated to between 400 and 1000 cc. It is to be noted that any other balloon may be used, e.g., a Foley catheter balloon or an angioplasty balloon, in the stomach or in any other suitable location in the body of the subject. These other. balloons may be filled to the capacity appropriate for the location of the body in which these other balloons may be implanted.

As shown, a catheter 40 is coupled to balloon 20 at a proximal portion 21 of balloon 20. Catheter 40 is typically configured to remain in stomach 22 of the subject following implantation of balloon 20, and to function as an anchor which restricts passage of the balloon, or any part of the implantable apparatus, through the pyloric sphincter (particularly if the balloon deflates while in the stomach).

As shown, system 18 is advanced into stomach 22 balloon-first. That is, balloon 20 is disposed at the distal-most end of system 18 during the advancement of system 18 into stomach 22 of the subject.

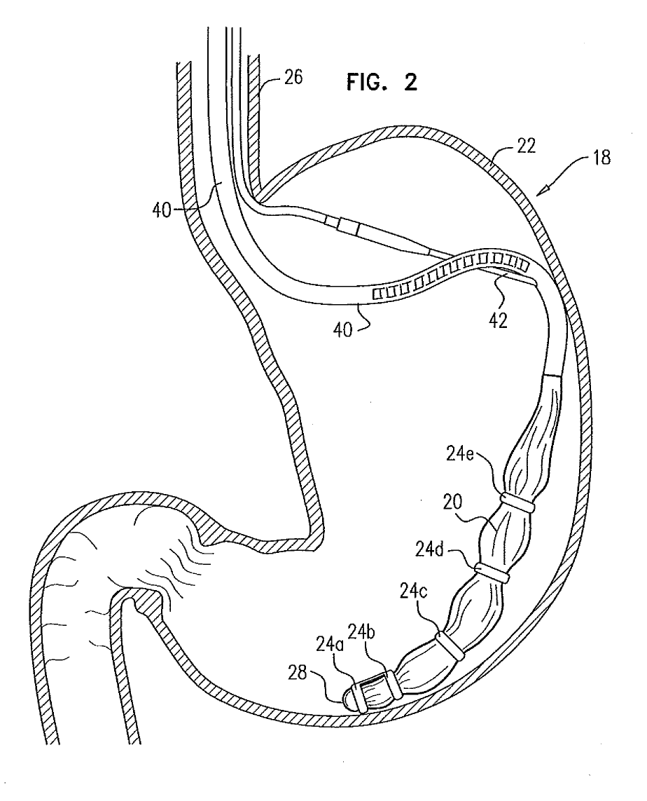

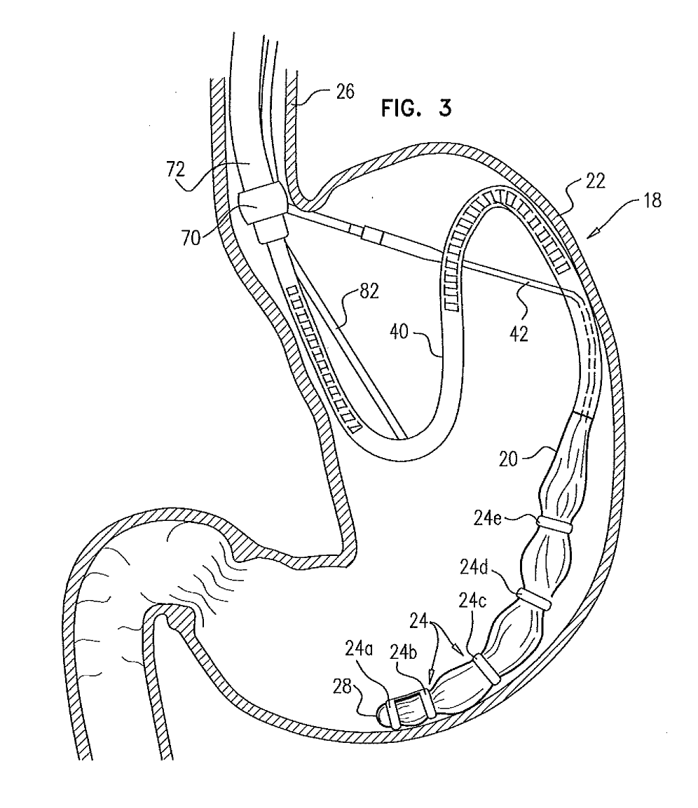

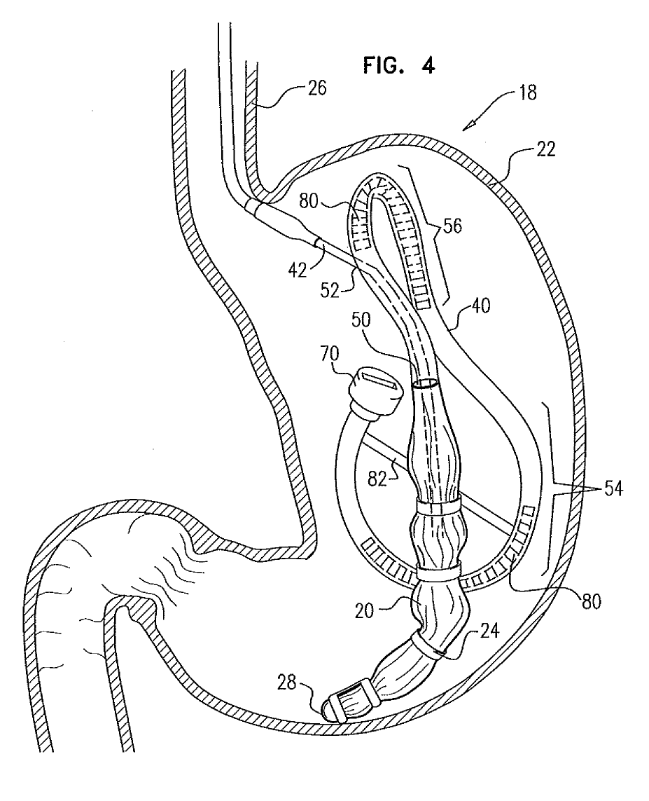

Reference is now made to FIG. 2, which is a schematic illustration of catheter 40 being advanced into the subject's stomach 22 proximally to balloon 20, in accordance with some applications of the present invention. For some applications, catheter 40, which functions as an anchor, is generally similar to the catheter (which also functions as an anchor) described with reference to FIG. 15 of US 2010/0121371 to Brooks, which is incorporated herein by reference. The function of the catheter is described in further detail hereinbelow, with reference to FIGS. 3-4. For some applications, a distal portion of a stretchable inflation tube 42 for inflating balloon 20 is disposed inside catheter 40. The distal portion of catheter 40, and/or inflation tube 42 typically extends within balloon 20 to distal end 28 of balloon 20. The distal-most end of inflation tube 42 is in fluid communication with the inside of balloon 20, such that fluid passes from inflation tube 42 into balloon 20. Inflation tube 42 exits catheter 40 at a site proximal to balloon 40 (e.g., within between 1 and 10 cm of proximal portion 21 of balloon 20), and a proximal portion of inflation tube 42 runs alongside catheter 40, as shown.

The portion of inflation tube 42 that is disposed within catheter 40 may be disposed within the central core of catheter 40 or may be disposed in a conduit channel (not shown) within the central core of catheter 40.

Catheter 40 is flexible and comprises a material, e.g., silicone. The proximal portion of catheter 40 (i.e., the portion of catheter 40 that extends proximally from balloon 20 and is not disposed within balloon 20), which excluding any straight portions thereof, has an elastic memory for assuming a pre-selected bent, or curved, configuration. In this pre-selected bent, or curved, configuration, catheter 40 functions as an anchor which prevents passage of balloon 20 through the pyloric sphincter of the subject.

Catheter 40 is shaped so as to define a proximal opening and central core extending from the opening toward the distal end of catheter 40. The distal end of catheter 40 may be opened or closed. The core of catheter 40 is configured to receive therethrough a removable straightening rod for straightening of catheter 40 from its pre-selected bent, or curved, configuration.

For some applications, the proximal portion of catheter 40 that is formed into the pre-selected bent, or curved, configuration has a diameter of between 4 and 20 cm, e.g., between 6 and 14 cm, when formed into the pre-selected bent, or curved configuration.

Typically, during the advancement of system 18 through esophagus 26 of the subject, the removable straightening rod (not shown for clarity of illustration) is disposed within the central core of catheter 40 so as to straighten and impart rigidity to catheter 40 as it is advanced through esophagus 26. As successive portions of catheter 40 are advanced into in stomach 22, the straightening rod is gradually removed from within the core of catheter 40 so as to enable catheter 40 to assume its pre-selected bent, or curved, configuration, as shown in FIG. 2. During the advancement of catheter 40 and balloon 20 into stomach 22, a proximal end of inflation tube 42 may be connected to an extension tube (not shown) which remains disposed outside the body of the patient.

Reference is now made to FIGS. 3-4, which are schematic illustrations of the deformation of catheter 40 into an anchor for anchoring the balloon hi the subject's stomach, in accordance with some applications of the present invention.

As described in US 2010/0121371 to Brooks, which is incorporated herein by reference, balloon 20 is typically sealed along a portion of catheter 40 in a vicinity of a distal end portion thereof. Catheter 40 is shaped to define a hollow lumen for passage therethrough of at least a portion of inflation tube 42. A distal portion of catheter 40 is disposed within balloon 20, while the remaining portion of catheter 40 is disposed outside balloon 20, Catheter 40 exits balloon 20 at only one location thereof, i.e., a proximal opening 50 (shown in FIG. 4) of balloon 20. In such an application, balloon 20 has only one opening, as shown.

Catheter 40 comprises a flexible, tubular material, e.g., silicone. The portion of catheter 40 that extends away from balloon 20 has an elastic memory for assuming a relaxed, pre-selected bent, or curved, configuration, as shown in FIG. 4. As shown in FIG. 4, the pre-selected bent, or curved, configuration has one or more “C” shaped curved portions (e.g., two “C” shaped curved portions 54 and 56, as shown) which form catheter 40 into a generally “S” shape. (The upper and lower curved portions of the “S” shape may be non-coplanar, as shown in FIG. 4, or coplanar.) Thus, the portion of catheter 40 that extends away from balloon 20, when assuming its pre-selected bent, or curved, configuration, functions as a planar anchor. It is to be noted that the pre-selected bent, or curved, configuration may be of any suitable shape, e.g., helical, “U”-, “S”-, or “C”-shaped.

Inflation tube 42 typically comprises an elastic material, e.g., silicone, PTFE, or ePTFE. Typically, a distal portion of inflation tube 42 is disposed within catheter 40 while the remaining portion of inflation tube 42 is disposed outside catheter 40. Inflation tube 42 exits catheter 40 though an opening 52 (shown in FIG. 4) in catheter 40 at a site proximal to proximal opening 50 of balloon 20, and, for some applications, distal to the curve of “U”-shaped portion 56 (shown in FIG. 4) of catheter 40.

Catheter 40 comprises an open proximal end having a central lumen extending toward its distal end. During the initial advancement and positioning of balloon 20 within the stomach of the subject, the central lumen of the catheter receives a straightening rod (not shown) therethrough and catheter 40 is straightened from its pre-selected bent shape, as shown in FIG. 1, for example. During advancement of the balloon into the subject's stomach, the balloon is the distal-most portion of the apparatus so as to facilitate atraumatic advancement of the apparatus toward the stomach. During the advancement, inflation tube 42 is advanced alongside and in parallel with catheter 40.

As shown in FIG. 3, a pushing member 72 is reversibly coupled to a proximal end 70 of catheter 40. The straightening rod is also advanceable through a lumen of pushing member 72. As catheter 40 is successively positioned within the stomach of the subject, the straightening rod is gradually removed, and thereby catheter 40 is allowed to assume its relaxed, pre-selected bent, or curved, configuration (as shown). Respective stages of the relaxation of the catheter are shown in FIGS. 3-4. This pre-selected bent, or curved, configuration enables catheter 40 to function as an anchor in order to prevent migration of the apparatus through the pyloric sphincter. Once proximal end 70 of catheter 40 is positioned in stomach 22, pushing member 72 is decoupled from proximal end 70 of catheter 40, and is removed from the subject. Proximal end 70 typically comprises a luer-lock port, or is sealed, so as to prevent the escape from and/or introduction of fluid within catheter 40.

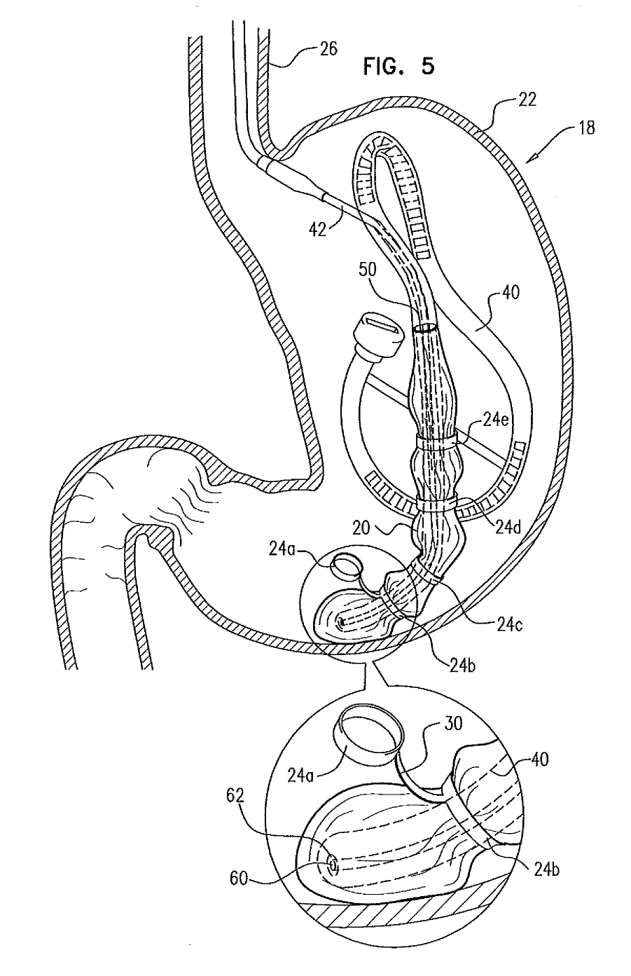

As catheter 40 assumes its bent configuration, the proximal portion of inflation tube 42 remains disposed outside catheter 40 in a resting position thereof. A distal end 60 (shown hereinbelow in FIG. 5) of inflation tube 42 is exposed from within catheter 40 through an opening 62 defined in the portion of catheter 40 that is disposed within balloon 20. Once balloon 20 is fully positioned within the stomach of the subject, fluid (e.g., air, gas such as helium or nitrogen, or liquid such as water or saline), foam, or any other filler known in the art is injected through inflation tube 42, in order to inflate balloon 20, for example, in accordance with the techniques described in PCT Application Publication WO 08/132745 to Brooks, which is incorporated herein by reference.

It is noted that the scope of the present invention includes using bands 24 in combination with a gastric balloon system having a different configuration from that described herein. For example, the scope of the present invention may include using bands 24 on a gastric balloon even in the absence of catheter 40. Furthermore, the scope of the present invention includes using bands 24 in combination with a gastric balloon system in which the inflation tube for inflating the balloon terminates at a different position along the balloon from the distal end of the balloon, for example, in the center of the balloon, mutatis mutandis, as described hereinbelow.

In FIG. 4, catheter 40 is shown in its pre-selected bent, or curved, configuration in which catheter 40 is shaped so as to define two “C”-shaped portions 54 and 56. In such a bent, or curved, configuration, catheter 40 functions as an anchor to prevent migration of system 18 through the pyloric sphincter muscle. A respective bend-limiting element 80 is disposed within catheter 40 at portions 54 and 56, and restricts bending of portions 54 and 56 beyond a predetermined amount. Bend-limiting elements 80 thus prevent catheter 40 from folding and being caught in the pyloric sphincter. For some applications, each bend-limiting element 80 (or another shape-controlling element) comprises a chain, i.e., a plurality of linked mechanical elements. For other applications, each bend-limiting element 80 comprises a plurality of mechanical elements that are not chained together but are disposed in series in a manner which limits back and forth flexing of the portion of catheter 40 in which bend-limiting element 80 is disposed. For other applications, each bend-limiting element 80 comprises a tube which is shaped so as to define a plurality of slits which permit the tube to assume a straight position, or to bend up to a predefined limit.

Typically, each bend-limiting element 80 permits limited back and forth flexing of the portion of catheter 40 in which bend-limiting element 80 is disposed.

Additionally, a cross-bar element 82 (or another shape-controlling element) is shown in a relaxed state thereof, bridging opposing portions of portion 54 of catheter 40 in a relaxed state thereof (FIG. 4). Although one cross-bar element 82 is shown, it is to be noted that two or more cross-bar elements may be coupled to catheter 40 at any location along portions 54 or 56 of catheter 40.

Cross-bar element 82 typically comprises a flexible material, e.g., silicone, such that during the advancement of system 18, cross-bar element 82 is typically stretched and aligned approximately in parallel with and alongside the straightened catheter 40 (FIG. 3). Once the straightening rod is removed from within catheter 40, cross-bar element 82 is allowed to assume its configuration as shown in FIG. 4 bridging portions of “C”-shaped element and opposite side.

Cross-bar element 82 helps maintain the shape of portion 54. It is to be noted that although system 18 shows cross-bar element 82 at portion 54, cross-bar element 82 or an additional cross-bar element may bridge opposing portions of portion 56.

Typically, bend-limiting element 80 prevents compression of portion 54 (and/or portion 56) of catheter 40 in response to a compression force applied to portion 54. Typically, cross-bar element 82 prevents (1) the opening of portion 54 (and/or portion 56) when balloon 20 and catheter 40 are inside the stomach, and (2) allows stretching of portion 54 (and/or portion 56) of catheter 40 during delivery into and removal from stomach 22 of the balloon 20 and catheter 40.

Reference is now made to FIG. 5, which is a schematic illustration of the distal end of balloon 20 being inflated, thereby causing the most distal band 24a of bands 24 to slide off the balloon, in accordance with some applications of the present invention. For some applications, due to the configuration of the bands on the balloon, and due to the fact that opening 60 of inflation tube 42 is distal to the distal band, inflation of the balloon is initiated at distal end 28 of the balloon.

As the distal end of the balloon becomes inflated, the most distal band 24a slides off the distal end of the balloon. Bridge 30, which couples the most distal band 24a to the next most distal band 24b, prevents the most distal band 24a from becoming decoupled from the apparatus, when the distal-most band 24a is displaced. For some applications, a different element is used to couple the most distal band 24a to the next most distal band 24b, another one or bands 24, or to another portion of system 18.

Alternatively, the most distal band is placed on the balloon such that, at the initiation of the inflation of the balloon, the band slides proximally along the balloon, in a similar manner to the other bands, as described hereinbelow (i.e., not sliding off balloon 20). For some applications, even though the most distal band 24 is configured to slide proximally along the balloon, the band is nevertheless coupled to the next most distal band, in order to prevent the most distal band from becoming decoupled from the apparatus.

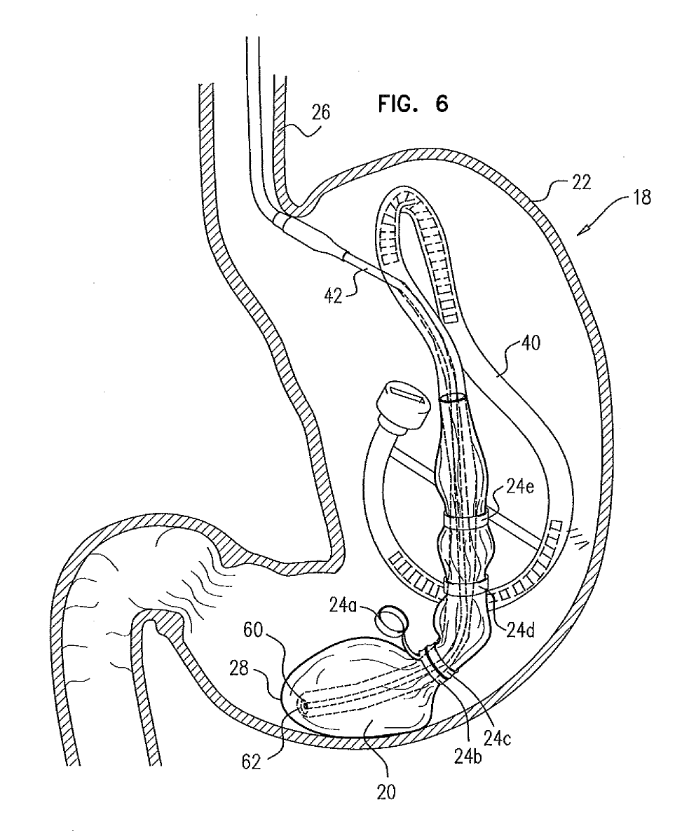

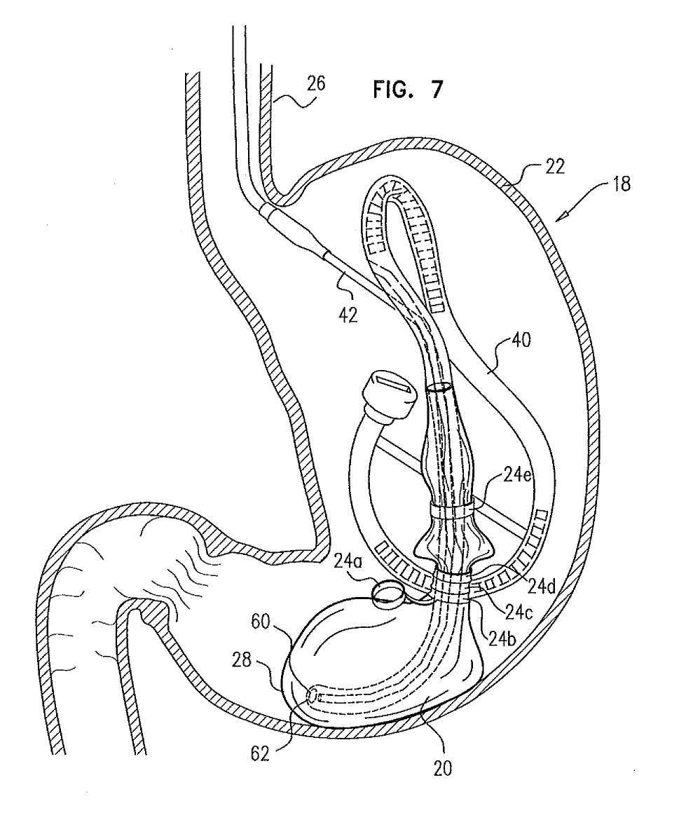

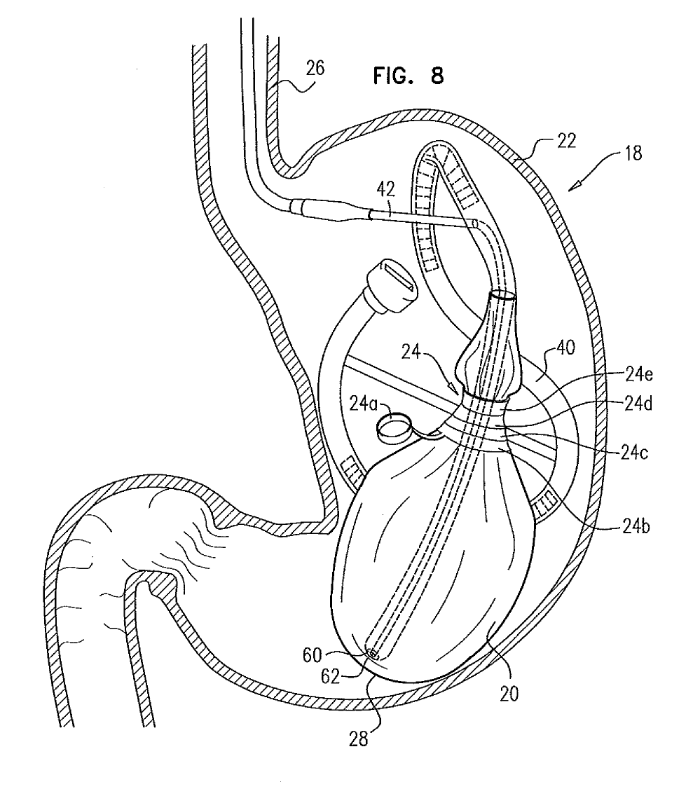

Reference is now made to FIGS. 6-9, which are schematic illustrations of respective bands 24 being slid proximally along balloon 20 at respective stages of the inflation of the balloon, in accordance with some applications of the present invention. As stated hereinabove, due to the configuration of the bands on the balloon, and due to the fact that opening 60 of inflation tube 42 is distal to the most distal band, inflation of the balloon is initiated at distal end 28 of the balloon. Furthermore, the expansion of the balloon continues to take place from the distal end of the balloon toward the proximal end of the balloon. Therefore, as balloon 20 continues to be inflated, the expanding balloon slides the remaining bands proximally along the balloon. That is, in response to initial inflation of the balloon, the distal-most band 24a slides proximally or slides off balloon 20, as shown. Then, in response to continued inflation of the balloon, the band 24b that is disposed adjacently proximally to the distal-most band slides, together with the distal-most band 24a toward the band 24c that is disposed adjacently proximally to band 24b. Then, band 24c slides together with bands 24a and 24b toward band 24d. Then, band 24d slides together with bands 24a, 24b, and 24c toward band 24e. Thus, the placement of the bands on the balloon and their successive proximal advancement facilitates controlled inflation of the balloon from the distal portion of balloon 20 toward the proximal portion of balloon 20.

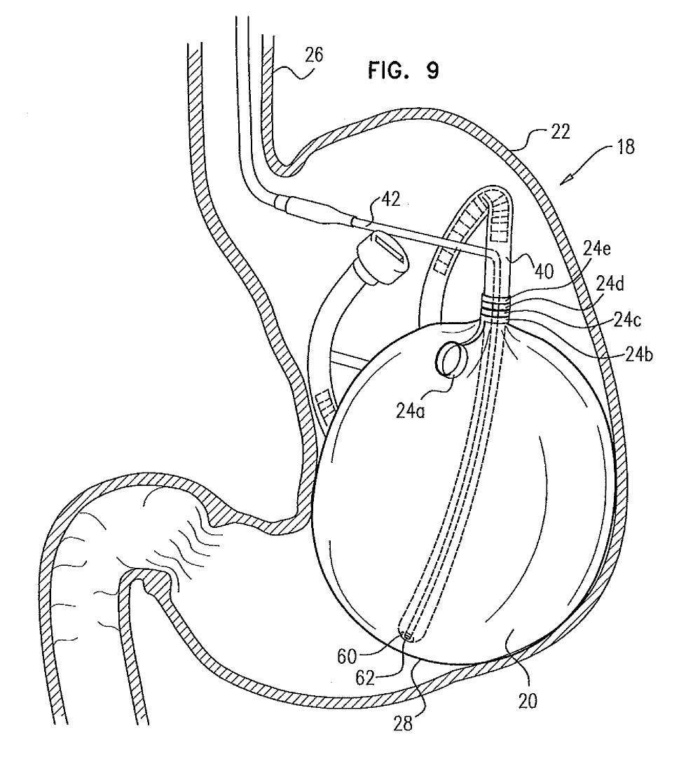

As shown in FIG. 9, inflation of the balloon causes all of bands 24 to slide onto catheter 40. Due to the elasticity of the bands, bands 24 become secured (or otherwise coupled) to catheter 40 (e.g., bands 24 return to their inner diameter of between 5 and 30 mm), thereby preventing bands 24 from becoming decoupled from the apparatus. For some applications, inflation of balloon 20 causes bands 24 to slide onto, and become secured to, a different portion of the apparatus, e.g., a portion that remains in the stomach. For example, bands 24 may be coupled to any portion of balloon 20 (e.g., proximal portion 21), or bands 24 may be coupled to a portion of inflation tube 42.

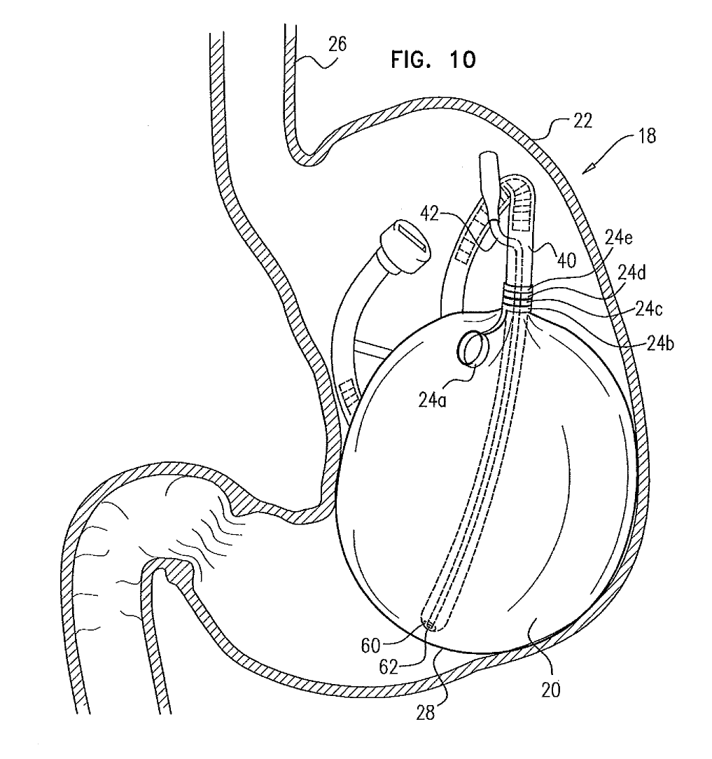

Reference is now made to FIG. 10, which shows system 18 within stomach 22 of the subject following inflation and following the return of inflation tube 42 within stomach 22. In this state, the esophagus of the subject is free from any of system 18. Following the initial inflation of balloon 20, balloon 20 may be further inflated or deflated when the physician endoscopically or non-endoscopically accesses inflation tube 42 and stretches inflation tube 42 (or an attachment couplable thereto) so that the proximal end of inflation tube 42 is exposed from within the body of the subject. Balloon 20 is then inflated further or deflated. As shown, catheter 40 remains in its bent, or curved, configuration in which catheter 40 functions as an anchor.

Typically, for some applications of the present invention, catheter 40 is decoupleable from balloon 20, and thus removal of catheter 40 from the subject removes bands 24 from the subject.

For some applications in which catheter 40 is not used, the bands may slide onto an inflation tube that is inserted directly into proximal portion 21 of the balloon. For some applications, such an inflation tube is decouplable from the balloon. Thus, subsequent to the bands sliding onto the inflation tube, the bands may be removed from the subject's body, by decoupling the inflation tube from the balloon, and removing the inflation tube from the subject's body.

For applications in which inflation tube 42 and catheter 40 remain coupled to balloon 20 (as shown in FIGS. 1-10), once the balloon is inflated, the physician releases the proximal end of inflation tube 42 so that it returns from its stretched state, within stomach 22 alongside catheter 40. The proximal end of inflation tube 42 is coupled to a Luer-lock port which prevents the escape of fluid from the balloon through tube 42 when balloon 20 is not being inflated.

The inflation of the balloon within the stomach promotes a feeling of satiety in the subject and generally interferes with peristaltic waves and gastric emptying.

During subsequent inflations of balloon 20, the proximal portion of inflation tube 42 is accessed endoscopically or non-endoscopically and is pulled and stretched from its resting position (i.e., alongside the rest of system 18), through esophagus 26, and toward the mouth of the subject. As it is pulled, inflation tube 42 is stretched (as shown in FIG. 9), and is pulled without pulling core catheter 40, which continues to maintain its pre-selected bent configuration during the pulling of inflation tube 42.

In a resting state of the highly-stretchable inflation tube 42 (i.e., when tube 42 is not pulled) a proximal portion of tube 42 protrudes from catheter 40, typically by a length of about 1-6 cm (e.g., about 2-4 cm). A length of a distal portion of tube 42 of about 6-12 cm (e.g., about 8-9 cm) remains within balloon 20. In an unstretched state, an outer resting diameter of tube 42 is typically about 2-4 mm, e.g., 3 mm. In some applications of the present invention, inflation tube 42 is stretched to more than 2.5 times and up to 10 times or more its resting position length (e.g., 6 times its resting position length) in order to facilitate inflation of balloon 20 from a site outside the body of the subject. In such an application, a proximal end of inflation tube 42 is disposed outside the body of the subject, while balloon 20 remains within stomach 22 of the subject. For some applications, the proximal portion of inflation tube 42 stretches to more than 2 times, 2.5 times, and up to 10 times or more its resting position length (e.g., 6 times its resting position length).

If it is not practical to inflate balloon 20 while inflation tube 42 is thus stretched, then a temporary filling tube (not shown) is coupled to a closure mechanism or to a temporary coupling valve, in order to provide fluid communication between the temporary filling tube and inflation tube 42. In such an application, inflation tube 42 is gradually returned to the stomach, substantially returning to its resting diameter, and the balloon is inflated by fluid passing through the temporary filling tube and inflation tube 42. Subsequently, the temporary filling tube is decoupled from inflation tube 42, and the closure mechanism is closed, in order to maintain the balloon in its newly inflated state.

As stated hereinabove, the scope of the present invention includes using bands 24 in combination with a gastric balloon system in which the distal end of the inflation tube for inflating the balloon terminates at a different position along the balloon from the distal end of the balloon, for example, in the center of the balloon, mutatis mutandis. For such applications, a portion of the bands may be slid distally along the balloon, as the balloon expands. Typically, the bands that advance distally are coupled to one or more of the other bands, for example, by a bridge that is similar to bridge 30, described hereinabove, with reference to FIG. 5. For such an application of the present invention, balloon 20 inflates from a generally longitudinal center thereof simultaneously in a distal and proximal direction.

For other applications, a distal end of the inflation tube for inflating the balloon terminates, at a proximal end of the balloon, mutatis mutandis. For such applications, bands 24 may be slid distally along the balloon, as the balloon expands. That is, for such an application of the present invention, balloon 20 inflates from a proximal portion thereof in a distal direction.

Reference is now made to FIGS. 1-10. It is to be further noted that the scope of the present invention includes the use of bands 24 with balloon 20 independently of catheter 40 and/or inflation tube 42. For applications in which balloon 20 comprises a gastric balloon, the balloon is capable of being inflated to between 400 and 1000 cc. It is to be noted that any other balloon may be used, e.g., a Foley catheter balloon or an angioplasty balloon, in the stomach or in any other suitable location in the body of the subject. These other balloons may be filled to the capacity appropriate for the location of the body in which these other balloons may be implanted.

Reference is again made to FIGS. 1-10. It is to be noted that balloon 20 and bands 24 may be provided without catheter 40 which functions as an anchor. That is, for some applications of the present invention, only balloon 20 and bands 24 remain in the stomach of the subject. For such an application, following the sliding of bands 24, bands 24 are coupled to any portion of balloon 20, e.g., proximal portion 21 of balloon 20. For such an application, an inflation tube is coupled to the balloon, (e.g., a stretchable inflation tube, as described herein). For some applications of the present invention, balloon 20, bands 24, and the inflation tube remain within the stomach of the subject. In such an application, following the sliding of bands 24, bands 24 may be coupled to any portion of balloon 20 (e.g., proximal portion 21 of balloon 20) or to a portion of the inflation tube.

It is to be yet further noted that the scope of the present invention includes the use of intrabody balloons other than gastric balloons, e.g., angioplasty balloons, a balloon configured to be placed in the biliary tract, a balloon configured to be placed in the vasculature of the subject, a balloon configured to treat an abscess, a balloon configured to treat a strictured area, a balloon configured to be placed in the urinary tract, a balloon configured to be placed in the digestive tract, Foley catheter balloons, or any other intrabody balloon known in the art.

The scope of the present invention includes embodiments described in one or more of the following;

US Patent Application Publication 2006/0142731 to Brooks, entitled, “Floating gastro-intestinal anchor,” filed May 18, 2005;

PCT Patent Publication WO 06/070361 to Brooks, filed Dec. 27, 2005, entitled “Floating gastrointestinal anchor”;

PCT Patent Publication WO 07/110866 to Brooks et al., entitled “Floating gastrointestinal anchor,” filed Mar. 28, 2007;

US Patent Application Publication 2009/0287231 to Brooks et al., entitled “Floating gastrointestinal anchor,” filed Mar. 28, 2007;

PCT Application Publication WO 08/132745, entitled “Non-endoscopic insertion and removal of device,” to Brooks, filed Apr. 30, 2008;

US Patent Application Publication 2010/0016871 to Brooks et al., entitled “Floating gastrointestinal anchor,” filed Feb. 26, 2009;

U.S. Provisional Application 61/250,888, entitled “Balloon hydraulic and gaseous expansion system,” to Hirszowicz, filed Oct. 13, 2009;

US Patent Application Publication 2010/0121371 to Brooks et al., entitled, “Non-endoscopic insertion and removal of a device,” filed Jan. 6, 2010; and/or

U.S. Provisional Application 61/329,643 to Hirszowicz et al., entitled, “Balloon hydraulic and gaseous expansion system,” filed Apr. 30, 2010.

All of the above-referenced applications are incorporated herein by reference. Techniques described herein can be practiced in combination with techniques described in one or more of the above-referenced applications.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
an intrabody balloon configured to be in a folded state during insertion into a subject's body, and to become inflated inside the subject's body; and
one or more bands disposed annularly around the balloon at respective longitudinal positions along the balloon, the bands configured:
while the balloon is in the folded state thereof, to secure the balloon in the folded state, and
due to the balloon becoming inflated, to slide and become coupled to a portion of the apparatus.

2. The apparatus according to claim 1, wherein the one or more bands comprise a plurality of bands, and wherein a band that is disposed most distally along the balloon with respect to other bands of the plurality of bands is coupled to at least one other band of the plurality of bands.

3. The apparatus according to claim 1, wherein the portion of the apparatus comprises a portion of the balloon, and wherein the one or more bands are configured to be coupled to the portion of the balloon.

4. The apparatus according to claim 1, wherein the balloon comprises a gastric balloon.

5. The apparatus according to claim 4, wherein the balloon comprises a 400-1000 cc balloon.

6. The apparatus according to claim 1, wherein each band of the one or more bands has an inner diameter of between 5 and 30 mm in the folded state of the balloon.

7. The apparatus according to claim 6, wherein each band has an inner diameter of between 10 and 20 mm in the folded state of the balloon.

8. The apparatus according to claim 6, wherein each band is configured to expand to between 15 and 150 mm during the inflation of the balloon.

9. The apparatus according to claim 1, wherein the one or more bands comprises two or more bands.

10. The apparatus according to claim 9, wherein the one or more bands comprises 5 to 7 bands.

11. The apparatus according to claim 1, further comprising an anchor coupled to a proximal portion of the balloon and configured to anchor the balloon inside the subject's body, wherein the bands are configured to become coupled to the anchor responsively to the inflating.

12. The apparatus according to claim 11, wherein the bands are configured to slide proximally toward the proximal portion of the balloon.

13. The apparatus according to claim 11, wherein the portion of the apparatus comprises the proximal portion of the balloon, and wherein the one or more bands are configured to be coupled to the proximal portion of the balloon.

14. The apparatus according to claim 11, wherein the portion of the apparatus comprises a portion of the anchor, and wherein the one or more bands are configured to be coupled to the portion of the anchor.

15. The apparatus according to claim 1, wherein the one or more bands comprise a plurality of bands disposed annularly around the balloon, at respective longitudinal positions along the balloon.

16. The apparatus according to claim 15, wherein the plurality of bands are expandable responsively to the inflation of the balloon.

17. The apparatus according to claim 15, wherein the plurality of bands facilitate controlled inflation of the balloon when, responsively to increasing levels of inflation of the balloon, a distal-most band of the plurality of bands is displaced and each remaining band of the plurality of bands slides proximally, in succession, to expose and facilitate unfolding of successive portions of the balloon.

18. The apparatus according to claim 1, further comprising a catheter coupled at a distal portion thereof to a proximal portion of the balloon.

19. The apparatus according to claim 18, wherein the bands are configured to slide proximally toward the proximal portion of the balloon.

20. The apparatus according to claim 18, wherein the portion of the apparatus comprises the proximal portion of the balloon, and wherein the one or more bands are configured to be coupled to the proximal portion of the balloon.

21. The apparatus according to claim 18, wherein the portion of the apparatus comprises a portion of the catheter that is proximal to the proximal portion of the balloon, and wherein the one or more bands are configured to be coupled to the portion of the catheter that is proximal to the proximal portion of the balloon.

22. The apparatus according to claim 18, wherein, in the folded state, a distal end of the balloon is rounded.

23. The apparatus according to claim 18, wherein the distal portion of the catheter is disposed within the balloon, and a proximal portion of the catheter extends away from the balloon.

24. The apparatus according to claim 23, wherein the balloon has an opening that is sealed around a portion of the catheter.

25. The apparatus according to claim 1, wherein the portion of the apparatus comprises an inflation tube, the balloon being configured to be inflated via the inflation tube, wherein the bands are configured to become coupled to the inflation tube responsively to the inflating.

26. The apparatus according to claim 25, wherein the inflation tube is configured to be decoupled from the balloon and to remove the bands from a body of the subject by the inflation tube being removed from the subject's body.

27. The apparatus according to claim 1, wherein the apparatus is configured to be positioned in an antrum of a stomach of a subject such that the apparatus to some extent interferes with natural antral contractions of the antrum.

28. A method, comprising:
providing apparatus that includes an intrabody balloon that is configured to be in a folded state during insertion into a subject's body, and to become inflated inside the subject's body; and
placing one or more bands annularly around the balloon at respective longitudinal positions along the balloon, such that:
while the balloon is in the folded state thereof, the bands secure the balloon in the folded state, and
due to the balloon becoming inflated, the bands slide proximally and become coupled to a portion of the apparatus.

29. The method according to claim 28, wherein placing the one or more bands annularly around the balloon comprises facilitating controlled inflation of the balloon when the balloon is inflated.

30. (canceled)

31. The method according to claim 28, wherein the portion of the apparatus includes a portion of the balloon.

32-37. (canceled)

38. The method according to claim 28, further comprising coupling a proximal portion of the balloon to a distal portion of a catheter, wherein:
coupling the proximal portion of the balloon to the distal portion of the catheter comprises coupling proximal portion of the balloon to the distal portion of a catheter in a manner in which the distal portion of the catheter is disposed within the balloon and a proximal portion of the catheter extends away from the balloon, and
the portion of the apparatus includes one or more portions selected from the group consisting of a portion of the proximal portion of the catheter and the proximal portion of the balloon.

39-66. (canceled)
